(19) United States
(12) Patent Application Publication
(10) Pub. No.: US 2009/0209923 A1
(43) Pub. Date: Aug. 20, 2009

(54) Disposable hypodermic needle

(76) Inventors: Sören Linderoth, Roskilde (DK); Alexander Van Lelieveld, Frederiksberg (DK)

Correspondence Address: HARNESS, DICKEY & PIERCE, P.L.C., P.O. BOX 8910, RESTON, VA, 20195, US

(21) Appl. No.: 11/918,858

(22) PCT Filed: Apr. 19, 2006
(86) PCT No.: PCT/DK2006/000208

§ 371 Date: Jan. 2, 2009

(30) Foreign Application Priority Data
Apr. 19, 2005 (DK) PA 2005 00569
Oct. 17, 2005 (DK) PA 2005 01458
Mar. 3, 2006 (DK) PA 2006 00321

(51) Int. Cl. A61M 5/32; C22F 1/00

(52) U.S. Cl. 604/272; 148/538; 148/561

(57) ABSTRACT

The present disclosure concerns (i) a hypodermic needle composed of a metal alloy, wherein the metal alloy is in a predominantly amorphous form, said amorphous form of said metal alloy having a glass transition temperature (T 9 ) in the range of 50-6500 C, (ii) methods for the manufacture of such injection needles by casting or moulding an amorphous alloy, and (iii) a method of safely disposing hypodermic needles.

### FIELD OF THE INVENTION

This invention relates to hypodermic needles which can be safely disposed after use.

### BACKGROUND OF THE INVENTION

Although sharp-edged hypodermic needles can be produced from steel, this material has significant disadvantages. Needles made from other hard materials such as carbides, sapphire or diamond would have a much higher manufacturing costs. For example, sharp-edged steel needles must be produced at high temperatures and cannot be disposed very easily. The good mechanical properties of steel and its high melting point (1400° C.) make it very hard to dispose in a safe manner, unless particularly designed containers are used. This put refuse workers and street cleaners at risk of life-threatening infections, such as hepatitis C and HIV, from syringes discarded by either legitimate needle users, including Type 1 diabetics, or intravenous drug users.

It has long been known that the primary engineering challenges for producing effective hypodermic needles are the shaping and manufacturing of a needle with a small cross-sectional area and an effective sharp edge in a cheap process.

Accordingly, there is a need for hypodermic needles having good mechanical properties, good processing properties and which are safe to dispose so as to eliminate the risk of life-threatening infections.

### SUMMARY OF THE INVENTION

One problem addressed by the present invention is the problem of disposing metal needles. Another problem is the easy manufacture of hypodermic needles.

Hence, the present invention provides a hypodermic needle composed of a metal alloy, wherein the metal alloy is in a predominantly amorphous form, said amorphous form of said metal alloy having a glass transition temperature (T_{g}) in the range of 50-650° C.

Thereby, a hypodermic needle is obtained which can be softened during heating by means of, e.g., a normal lighter and then be deformed in order to remove sharp edges or points, whereafter the needle can be disposed causing no risk for the personnel who accordingly are going to remove the disposal.

Hence, the present invention also provides a method of disposing a hypodermic needle as defined herein, the method comprising the steps of (i) heating at least the tip of said hypodermic needle to a temperature at or above the glass-transition temperature (T_{g}) of said metal alloy.

Furthermore, the hypodermic needle material has very good mechanical properties, is mouldable at a fairly low temperature (e.g. up to 650° C.), and renders it possible to shape the material into a sharp edged needle.

Thus, the present invention also provides
(i) a method of manufacturing a hypodermic needle, the method comprising the steps of
a. providing a feedstock of a molten liquid metal alloy,
b. casting the feedstock into the desired shape in form of a needle while cooling the metal alloys so as to bring said metal alloy into a predominantly amorphous form, said amorphous form of said metal alloy having a glass transition temperature (T_{g}) in the range of 50-650° C., and
c. optionally processing the needle to form a preliminary edge; and
(ii) a method of manufacturing a hypodermic needle, the method comprising the steps of
a. providing a feedstock of a solid piece of a metal alloy, said metal alloy being in a predominantly amorphous form, said amorphous form of said metal alloy having a glass transition temperature (T_{g}) in the range of 50-650° C.,
b. heating the feedstock to or above the glass transition temperature (T_{g}), but below the crystallisation temperature (Tₓ), of said metal alloy,
c. moulding the alloy into the desired shape in form of a needle, and
d. optionally processing the needle to form a preliminary edge.

### BRIEF DESCRIPTION OF THE DRAWINGS

FIG. 1 is a sketch of a needle with a 3-cut design.

FIG. 2 shows a flow-chart of a process for making needles shown in FIG. 1.

FIG. 3 shows a sketch of a needle with a new tip design.

### DETAILED DESCRIPTION OF THE INVENTION

The present invention provides novel hypodermic needles which can be safely disposed. More particularly, the invention provides a hypodermic needle composed of a metal alloy, wherein the metal alloy is in a predominantly amorphous form, said amorphous form of said metal alloy having a glass transition temperature (T_{g}) in the range of 50-650° C.

With respect to the expression “predominantly amorphous”, it is noted that the amorphous form of the metal alloy typically constitutes more than 75%, e.g. more than 80%, such as more than 85%, preferably more than 90%, e.g. 80-100%, by volume of said metal alloy.

The terms “hypodermic needle” is intended to mean a hollow needle commonly used with a syringe to inject substances into the body. A hypodermic needle may also be used to take liquid samples from the body, for example taking blood from a vein in venipuncture.

A hypodermic needle is typically in the form of an elongate tube or cannula having a fluid-conducting lumen and characterized by a central axis. The proximal end of the hypodermic needle is typically configured for mating to, being part of, or being otherwise affixed to, a fluid delivery device such as a hypodermic syringe. The distal end of the hypodermic needle is typically provided with a pointed tip geometry for piercing elastomeric septums and/or a patient's flesh or tissue so as to deliver the medicament held in the syringe. The practitioner may also employ the hypodermic needle for aspirating fluids held in a vessel such as a vial. This use often entails a practitioner inserting the pointed tip of the needle through a rubber or elastomeric-type seal associated with the vessel so that the practitioner can access the fluid contained within the vessel.

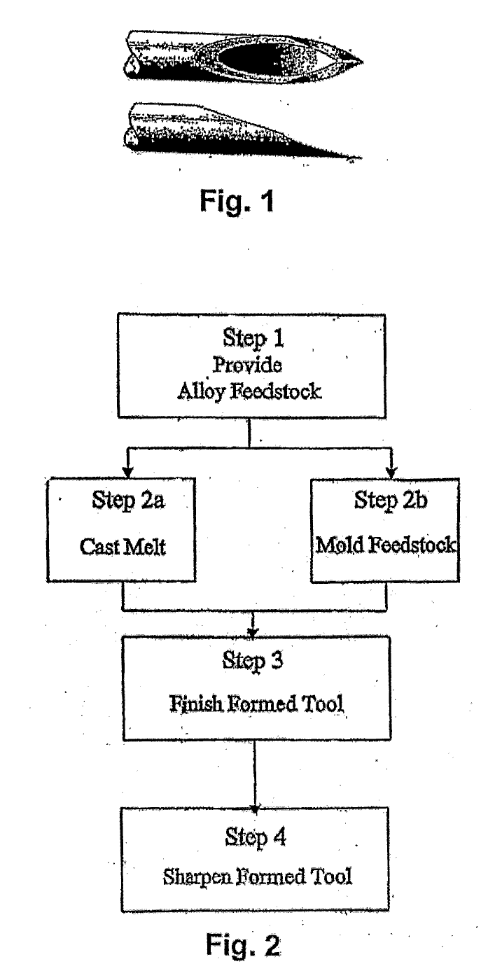

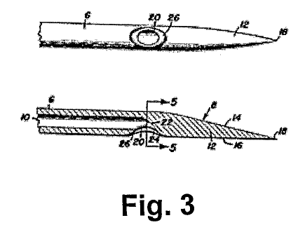

Examples of the geometry of the hypodermic needle are illustrated in FIGS. 1 and 3.

The hypodermic needle is composed of predominantly amorphous metal alloy, also referred to as “bulk amorphous alloy” in the following. A characteristic property of bulk amorphous alloys is that there exist a glass transition temperature (T_{g}) at a temperature below the temperature at which the amorphous alloy crystallises (Tₓ).

As it will be understood from the following, the glass transition temperature (T_{g}) plays an important role for the ease of manufacture and the safe disposal of the hypodermic needles described herein. In preferred embodiments, the glass transition temperature (T_{g}) is in the range of 80-650° C., such as in the range of 80-500° C. or in the range of 100-650° C., or in the range of 100-500° C., or in the range of 150-500° C., preferably in the range of 200-500° C. A glass transition temperature according to the above temperature intervals ensures that the hypodermic needle can be safely disposed after use. It also renders it possible to utilize conventional tools/moulds in the manufacturing process, see further below.

The temperature interval, ΔT, between the crystallisation temperature (Tₓ) and the glass transition temperature (T_{g}) of the metal alloy should typically be at least 5 K wide, and is often called the supercooled liquid region, because in this region, the alloy acts liquid-like and may easily be deformed. In most preferred embodiments, the temperature interval, ΔT, between the crystallisation temperature (Tₓ) and the glass transition temperature (T_{g}) of the metal alloy is at least 5 K, e.g. at least 20 K, such as in the range of 5-150 K, e.g. in the range of 10-150 K, or in the range of 20-120 K, or in the range of 30-100 K.

The bulk amorphous alloys cover a whole range of alloys with different properties. When using amorphous alloys to make a hypodermic needle, mechanical properties should be considered very careful. The most important mechanical properties to evaluate when choosing an alloy are brittleness and how easy the alloy is bend. The brittleness can be described in mechanical properties such as fracture toughness and elastic deformation strain limit. A high fracture toughness K_{IC} (>20 MPa·m^{1/2}) characterises a material that has low tendency to break under impact. Since many known amorphous alloys have a low fracture toughness, they tend to be brittle as a ceramic material. This is important since breaking a needle during use may cause serious injury to the user. A high elastic deformation strain limit of 2% or more is preferred and characterises a material that undergoes a deformation and returns to its initial shape. A high fracture deformation strain limit of 2% or more is preferred and characterises a material that can undergo a deformation, without the material fractures. This is important since the needle tip undergoes considerably stress during puncturing of the skin and should be able to return to its original shape.

How easy the alloy is bent can be described by the Young's modulus, which preferably should be higher than 20 GPa in order for the needle not to bend during puncturing of the skin. In comparison, stainless steel can have a Young's modulus of 200 GPa and although it is known that wood (7-14 GPa) and glass (100-120 GPa) have the ability to penetrate skin a high Young's modulus is more preferable at least 30 GPa, e.g. at least 50 GPa.

Preferably, the hypodermic needle is designed so as to undergo plastic deformation at strain levels of at least about 1.2%, e.g. at strain levels of at least about 2.0%.

Because the very low amount of material (5-10 mg) used to make a hypodermic needle, bulk prices of alloys may not be of particular importance.

The effect, where the alloy acts liquid-like, is also termed superplasticity. In the supercooled liquid region (ΔT), the material may be deformed many thousand percent without failure.

In some embodiments, the metal alloy of the hypodermic needle is anodized.

As mentioned above, a hypodermic needle made from amorphous alloys has the potential to provide sharp needles having high hardness, ductility, elastic limit and corrosion resistance. These properties can provide a sharp hypodermic needle that will not become as easily dull as a needle made out of conventional metals, e.g. stainless steel. In many situations two needles are needed in connection with one injection, where one needle is used to penetrate the rubber protecting the medicine in a container from which the medicine in drawn, followed by a needle change before injection into the patient. This exchange is needed because the first action makes the needle dull.

For some applications a hypodermic needle, that keeps its sharpness longer will be beneficial for examples for a diabetes patient, who might use a needle multiple times or in situations were new needles are difficult to come by. It is beneficial for patients taking hormones in relation to artificial insemination, where the patient shall take medicine every day for a longer period. The patient would benefit from improved needles which do not have to be exchanged every time. The reduction of exchanges will reduce the risk connected with this exchange of needle, it will be less costly, and there are greatly reduced problems related to the needle waste. Also, fewer needles are needed.

Conventional hypodermic needles are result of the difficult process of shaping stainless steel into a needle. Using amorphous alloys more flexibility can be introduced into the needle design, because of the low process temperature and easy moulding ability of these alloys. One particular design is disclosed in U.S. Pat. No. 2,634,726 where the needle hole is place on the side of the needle. By moving the needle hole to the side of the needle, the likelihood of clogging of the needle bore is minimized, so that fine particles (e.g. a suspended drug) might accompany the solution into the blood stream. This is a major problem since most medicine is stored under sterile conditions with a rubber septum to protect the medicine from bacteria and unwanted particles. Furthermore a normal needle design the needle hole cores the skin, instead of letting the skin slide along the needle when pierced into the skin. By arranging the needle hole as outlined in FIG. 3, a much better result can be obtained, since the skin will slide along the needle without being cut off by the needle hole. This will result in a real advantage for the patient providing less pain when injecting the needle into the body.

Extended openings along the side of the needle can provide the possibility to inject much faster medicine or other fluid, even with a thin needle. This will be beneficial for e.g. psychiatric patients who are given large doses of medicine into the muscles in order to release this medicine slowly over a long period. The same holds for e.g. medicine against yellow fever.

Other new and beneficial designs could include hooked needles for injecting into difficult reachable places. Bent hypodermic needles are today, e.g., used by cancer patients which need a continuous injection, but because the injection in the breast can damage the lungs the hypodermic needle is bent to reduce the injection depth. Such can be made easily using the amorphous alloys.

### Examples of Suitable Metal Alloys

Generally, bulk solidifying amorphous alloys refer to the family of amorphous alloys that can be cooled at cooling rates of as low as 500 K/sec or less, and retain their amorphous atomic structure substantially. Such bulk amorphous alloys can be produced in thicknesses of 1.0 mm or more, substantially thicker than conventional amorphous alloys having a typical cast thickness of 0.020 mm, and which require cooling rates of 10⁵ K/sec or more.

In view of the above, it has been found that particularly interesting metal alloys are those selected from the group consisting of:
a. Copper containing alloys,
b. Platinum containing alloys,
c. Palladium containing alloys,
d. Zirconium containing alloys, and
e. Titanium containing alloys.

More particular, the metal alloy is advantageously selected from the group consisting of:
a. copper containing alloys of the approximate formula CuₓZr_{y}Al_{z}YₚTi_{q}, wherein x=40-70, y=25-55, z=5-10,p=0-5, q=0-5, and the sum x+y+z+p+q is 100;
b. platinum containing alloys of the approximate formula PtₓCu_{y}Ni_{z}Pₚ, wherein x=55-60, y=13-17, z=3-7, p=20-25 and the sum x+y+z+p is 100;
c. palladium containing alloys of the approximate formula PdₓCu_{y}Ni_{z}FeₚP_{q}, wherein x=32-38, y=27-33, z=8-12,p=3-7, q=18-22 and the sum x+y+z+p+q is 100;
d. zirconium containing alloys of the approximate formula ZrₓAl_{y}Ti_{z}CuₚNi_{q}, wherein x=49-56, y=8-12, z=3-7, p=16-20, q=13-17 and the sum x+y+z+p+q is 100; and
e. titanium containing alloys of the approximate formula Ti_{41.5}Zr_{2.5}Hf₅Cu_{42.5}Ni_{7.5}Si₁ wherein x=39-45, y=1.5-3.5, z=3-7, p=40-45, q=6-9, r=0.8-1.2 and the sum x+y+z+p+q+r is 100.

When used herein, the expression “approximate formula” refers to the fact that the elements explicitly mentioned in the formula need not to form an exclusive list of elements. Thus, it is envisaged that trace amounts, i.e. up to 4% of the weight of the metal alloy, may be present.

Specific examples of useful metal alloy are those selected from the group consisting of:
a. copper containing alloys of the approximate formula Cu_{47.5}Zr_{47.5}Al₅, which has a Young's modulus of 87 GPa, elastic strain limit 2% and a fracture strain limit of 18%, T_{g} of 425° C. and ΔT>50° C.,
b. platinum containing alloys of the approximate formula Pt_{57.5}Cu_{14.7}Ni_{5.3}P_{22.5}, which has a Young's modulus of 95 GPa, elastic strain limit 1.5% and a fracture strain limit of 20%, and very high fracture toughness, approximately 80 MPa m^{1/2}. T_{g} of 235° C. and Δ>50° C.,
c. palladium containing alloys of the approximate formula Pd₃₅Cu₃₀Ni₁₀Fe₅P₂₀, which has a Young's modulus of 120 GPa, T_{g} of 298° C. and ΔT of 66° C.,
d. zirconium containing alloys of the approximate formula Zr_{52.5}Al₁₀Ti₅Cu_{17.9}Ni_{14.6}, which has a yield strength of 1700 MPa, an elastic strain limit of 2 to 2.2% and a fracture strain limit of 2-2.2%, Young's modulus of 90 GPa and a fracture toughness of 55 to 60 MPa·m^{1/2}, T_{g} of 410° C. and ΔT=90° C., and
e. titanium containing alloys of the approximate formula Ti_{41.5}Zr_{2.5}Hf₅Cu_{42.5}Ni_{7.5}Si₁, which has a Young's modulus of 100 GPa, T_{g} of 407° C. and ΔT>50° C.

Although specific bulk solidifying amorphous alloys are described above, it is believed that any suitable bulk amorphous alloy may be used which can sustain strains up to 1.2%, such as up to 1.5%, or more without any permanent deformation or breakage; and/or have a high fracture toughness of about 10 MPa·m^{1/2}or more, and more specifically of about 20 MPa·m^{1/2}or more; and/or have high hardness values of about 4 GPa or more, and more specifically about 5.5 GPa or more. In comparison to conventional materials, suitable bulk amorphous alloys have yield strength levels of up to about 2 GPa and more, exceeding the current state of the Titanium alloys. In addition to desirable mechanical properties, bulk solidifying amorphous alloys typically exhibit a very good corrosion resistance.

This being said, it is believed that a wider range of metal alloys may be used for the hypodermic needle of the present invention. Exemplary embodiments of suitable amorphous alloys are disclosed in U.S. Pat. Nos. 5,288,344; 5,368,659; 5,618,359; and 5,735,975; all of which are incorporated herein by reference.

One exemplary family of suitable bulk solidifying amorphous alloys are described by the following molecular formula: (Zr, Ti)ₐ (Ni, Cu, Fe)_{b} (Be, Al, Si, B)_{c}, where a is in the range of from about 30 to 75, b is in the range of from about 5 to 60, and c in the range of from about 0 to 50 in atomic percentages. It should be understood that the above formula by no means encompasses all classes of useful bulk amorphous alloys. For example, such bulk amorphous alloys can accommodate substantial concentrations of other transition metals, up to about 20% atomic percentage of transition metals such as Nb, Cr, V, Co. One exemplary bulk amorphous alloy family is defined by the molecular formula: (Zr, Ti)ₐ (Ni, CU)_{b}(Be)_{c}, where a is in the range of from about 40 to 75, b is in the range of from about 5 to 50, and c in the range of from about 5 to 50 in atomic percentages. One exemplary bulk amorphous alloy composition is Zr₄₁Ti₁₄Ni₁₀Cu_{12.5}Be_{22.5}. Yet another example is Zr_{52.5}Al₁₀Ti₅Cu_{17.9}Ni_{14.6} which has T_{g} of 683 K.

Another set of bulk-solidifying amorphous alloys are compositions based on ferrous metals (Fe, Ni, Co). Examples of such compositions are disclosed in U.S. Pat. No. 6,325,868, (A. Inoue et. al., Appl. Phys. Lett., Volume 71, p 464 (1997)), (Shen et. al., Mater. Trans., J M, Volume 42, p 2136 (2001)), and Japanese patent application 2000126277 (Publ. No. 2001303218 A), incorporated herein by reference. One exemplary composition of such alloys is Fe₇₂Al₅Ga₂P₁₁C₆B₄. Another exemplary composition of such alloys is Fe₆₀Co₈Zr₁₀Mo₅W₂B₁₅ with T_{g} of 898 K. Yet another set of ferrous metals bulk-solidifying amorphous alloys are compositions based on ferrous metals (Fe, Co). The composition can be given as (Co, Fe)ₐ (Ta)_{b} (B)_{c}, wherein “a” is in the range of from about 40 to 75, “b” is in the range of from about 2 to 15, and “c” in the range of from about 5 to 25 in atomic percentages. Although, these alloy compositions are not as processable as Zr-base alloy systems, these materials can be still be processed in thicknesses around 0.5 mm or more, sufficient enough to be utilized in the current disclosure. In addition, although the density of these materials is generally higher, from 6.5 g/cm³ to 8.5 g/cm³, the hardness of the materials is also higher, from 7.5 GPA to 12 GPa or more making them particularly attractive. Similarly, these materials have elastic strain limit higher than 1.2% and very high yield strengths from 2.5 GPa to 4 GPa.

Yet another set of bulk-solidifying amorphous alloys are compositions based on platinum and ferrous metals (Pt, Ni, Co). The composition can be given as (Pt)ₐ (Cu, Ni)_{b} (P, B, Si)_{c}, wherein “a” is in the range of from about 45 to 75, “b” is in the range of from about 15 to 30, and “c” in the range of from about 15 to 30 in atomic percentages. One exemplary composition of such alloys is Pt_{57.5}Cu_{14.7}Ni_{5.3}P_{22.5}. These materials can be processed in thicknesses around 0.5 mm or more and has T_{g} of 508 K, sufficient to be utilized in the current disclosure. These materials have elastic strain limit higher than 1.2%.

Yet another set of bulk-solidifying amorphous alloys are compositions based on Palladium (Pd). The composition can be given as (Pd)ₐ (Cu, Ni)_{b} (P, B)_{c}, wherein “a” is in the range of from about 30 to 50, “b” is in the range of from about 30 to 50, and “c” in the range of from about 15 to 25 in atomic percentages. One exemplary composition of such alloys is Pd_{42.5}Cu_{27.5}Ni₁₀P₂₀. These materials can be processed in thicknesses around 0.5 mm or more and has T_{g} of 572 K, sufficient to be utilized in the current disclosure. These materials have elastic strain limit higher than 1.2%. Another example is a bulk amorphous alloy characterised by the molecular formula (Pd)ₐ (Cu, Ni)_{b}(P, B, Si)_{c} where “a” is in the range of about 35 to 85, “b” is in the range of about 2 to 50, and “c” is in the range of about 10 to 30 in atomic percentages.

Yet another set of bulk-solidifying amorphous alloys are compositions based on Lanthanum (La). The composition can be given as (La, Ce, Pr, Nd)ₐ (Al, Si, B)_{b} (Cu, Ni, Fe)_{c}, wherein “a” is in the range of from about 45 to 70, “b” is in the range of from about 15 to 40, and “c” in the range of from about 15 to 30 in atomic percentages. One exemplary composition of such alloys is La₅₅Al₂₅Cu₂₀. These materials can be processed in thicknesses around 0.5 mm or more and has T_{g} of 456 K, sufficient to be utilized in the current disclosure. These materials have elastic strain limit higher than 1.2%.

Yet another set of bulk-solidifying amorphous alloys are compositions based on Neodymium (Nd). The composition can be given as (Nd)ₐ (Al, Si)_{b} (Ni, Cu, Fe, Co)_{c}, wherein “a” is in the range of from about 45 to 75, “b” is in the range of from about 5 to 20, and “c” in the range of from about 15 to 35 in atomic percentages. One exemplary composition of such alloys is Nd₆₁Al₁₁Ni₈Co₅Cu₁₅. These materials can be processed in thicknesses around 0.5 mm or more and has T_{g} of 445 K, sufficient to be utilized in the current disclosure. These materials have elastic strain limit higher than 1.2%.

Yet another set of bulk-solidifying amorphous alloys are compositions based on Cupper (Cu). The composition can be given as (Cu)ₐ (Zr, Ce, Hf, Ti)_{b} (Be, B)_{c}, wherein “a” is in the range of from about 50 to 75, “b” is in the range of from about 20 to 60, and “c” in the range of from about 0 to 25 in atomic percentages. One exemplary composition of such alloys is Cu₆₀Zr₃₀Ti₁₀. These materials can be processed in thicknesses around 0.5 mm or more and has T_{g} of 713 K, sufficient to be utilized in the current disclosure. These materials have elastic strain limit higher than 1.2%.

Yet another set of bulk-solidifying amorphous alloys are compositions based on Titanium (Ti). The composition can be given as : (Ti)ₐ (Ni, Cu)_{b} (B, Si, Sn, P)_{c}, wherein “a” is in the range of from about 40 to 65, “b” is in the range of from about 30 to 60, and “c” in the range of from about 5 to 25 in atomic percentages. One exemplary composition of such alloys is Ti₅₀Ni₂₄Cu₂₀B₁Si₂Sn₃. These materials can be processed in thicknesses around 0.5 mm or more and has T_{g} of 726 K, sufficient to be utilized in the current disclosure. These materials have elastic strain limit higher than 1.2%.

In general, crystalline precipitates in bulk amorphous alloys are highly detrimental to their properties, especially to the toughness and strength, and as such generally preferred to a minimum volume fraction possible. However, there are cases in which ductile metallic crystalline phases precipitate in-situ during the processing of bulk amorphous alloys. These ductile precipitates can be beneficial to the properties of bulk amorphous alloys especially to the toughness and ductility. Accordingly, bulk amorphous alloys comprising such beneficial precipitates are also included in the current invention, however still taking into account that the metal alloy must be in a predominantly amorphous form. One exemplary case is disclosed in (C. C. Hays et. al, Physical Review Letters, Vol. 84, p 2901, 2000), which is incorporated herein by reference.

Conventional materials, such as stainless steel, have a poly-crystalline atomic structure, which is composed of small crystalline grains oriented in varying orientations. Because the different grains in the material respond differently to the shaping operations, as such, the shaping and manufacture of highly effective sharp edges from such crystalline materials are either compromised or require significant additional processing raising the cost of the finished needle. Because bulk solidifying amorphous alloys do not have a crystalline structure, they respond more uniformly to conventional shaping operations, such as lapping, chemical, and high energy methods.

Because of the small radius of curvature of the tip edges of these needles, the edges have a low degree of stiffness, and are therefore subject to high levels of strain during injection through skin. For example, cutting edges made of conventional metals, such as stainless steel, sustain large strains only by plastic deformation hence losing their sharpness and flatness. In fact, conventional metals start deforming plastically at strain levels of 0.6% or less. On the other hand, cutting edges made of hard materials, such as diamond, do not deform plastically, instead they chip off due to their intrinsically low fracture toughness, as low as 1 or less ksi/sqrt (in), which limits their ability to sustain strains over 0.6%. In contrast, due to their unique atomic structure amorphous alloys have an advantageous combination of high hardness and high fracture toughness. Therefore, cutting blades made of bulk solidifying amorphous alloys can easily sustain strains up to 2.0% without any plastic deformation or chip-off. Further, the bulk amorphous alloys have higher fracture toughness in thinner dimensions (less than 1.0 mm) which makes them especially useful for sharp-edge needles.

### Method of Manufacture

In a further aspect, the invention also provides various methods for the manufacture of hypodermic needles.

FIG. 2 shows a flow-chart for a process of forming the amorphous alloy articles of the invention comprising: providing a feedstock (Step 1), in the case of a moulding process, this feedstock is a solid piece in the amorphous form, while in the case of a casting process, this feedstock is a molten liquid alloy above the melting temperatures; then either casting the feedstock from at or above the melt temperature into the desired shape while cooling (Step 2a), or heating the feedstock to the glass transition temperature or above and molding the alloy into the desired shape (Step 2b). Any suitable casting process may be utilized in the current invention, such as, permanent mold casting, die casting, extrusion moulding or a continuous process such as planar flow casting. One such die-casting process is disclosed in U.S. Pat. No. 5,711,363, which is incorporated herein by reference. Likewise, a variety of moulding operations can be utilized, such as, blow moulding (clamping a portion of feedstock material and applying a pressure difference on opposite faces of the unclamped area), injection moulding/die-forming (forcing the feedstock material into a die cavity), and replication of surface features from a replicating die. U.S. Pat. Nos. 6,027,586; 5,950,704; 5,896,642; 5,324,368; 5,306,463; (each of which is incorporated by reference in its entirety) disclose methods to form moulded articles of amorphous alloys by exploiting their glass transition properties. Although subsequent processing steps may be used to finish the amorphous alloy articles of the current invention (Step 3), it should be understood that the mechanical properties of the bulk amorphous alloys and composites can be obtained in the mould as cast and/or moulded form without any need for subsequent process such as heat treatment or mechanical working. In addition, in one embodiment the bulk amorphous alloys and their composites are formed into complex near-net shapes in the two-step process. In such an embodiment, the precision and near-net shape of casting and mouldings is preserved.

Finally, the needles are most often roughly machined to form a preliminary edge and the final sharp edge is produced by one or more combinations of the conventional lapping, chemical and high energy methods (Step 4).

One aspect of the invention relates to a method of manufacturing a hypodermic needle, the method comprising the steps of
a. providing a feedstock of a molten liquid metal alloy,
b. casting the feedstock into the desired shape in form of a needle while cooling the metal alloys so as to bring said metal alloy into a predominantly amorphous form, said amorphous form of said metal alloy having a glass transition temperature (T_{g}) in the range of 50-650° C., and
d. optionally processing the needle to form a preliminary edge.

Another aspect of the invention relates to a method of manufacturing a hypodermic needle, the method comprising the steps of
a. providing a feedstock of a solid piece of a metal alloy, said metal alloy being in a predominantly amorphous form, said amorphous form of said metal alloy having a glass transition temperature (T_{g}) in the range of 50-650° C.,
b. heating the feedstock to or above the glass transition temperature (T_{g}), but below the crystallisation temperature (Tₓ), of said metal alloy,
c. moulding the alloy into the desired shape in form of a needle, and
d. optionally processing the needle to form a preliminary edge.

In the before-mentioned method of manufacture, the moulding being conducted by means of an injection moulding machine.

The specifications with respect to the hypodermic needle and the amorphous metal alloy (bulk amorphous alloy) are preferably as described hereinabove.

Many relevant processes can be used to manufacture the hypodermic needles. Two illustrative processes are outlined in the following.

The needles can be produced by extrusion. The steps mentioned below outline a process of forming the amorphous alloy articles of the invention using extrusion:
Step 1: Providing a feedstock of amorphous alloy that is heated to the glass transition temperature or slightly above.
Step 2: A tube is extruded.
Step 3: The tube is cut into smaller pieces.
Step 4: The needles are sharpened with 3 way cut followed by grinding the edges.
Step 5: The needles are attached to a hub.

The needles could be produced by injection moulding. The steps mentioned below outline two processes of forming the amorphous alloy articles of the invention using injection moulding:

### 1. Over Wire Moulding

A continuous wire strand is used to form the needle bore channel by holding it central to the tool-molding channel. An injection moulding can be designed so that it can be modified to include a wire strand through its core. As each moulding is formed they are held on the wire string to be removed at a later date.

The process steps for injection moulding over a wire are:
1. With tool open, previous moulding ejected and wire clamped across tool cavity
2. Close tool
3. Inject amorphous alloy to form needle
4. Open to cavity
5. Release wire clamp near needle tip and eject component
6. Reclamp wire behind component and position wire across tool cavity ready for next cycle.

### 2. Over Pen Moulding

The process steps for over pen moulding are:
1. Close mould tool
2. Inject material into mould cavity around a heated core pin to prevent full solidification of the needle shaft.
3. Withdraw the core pin
4. Open the tool cavity whilst holding the end of the needle shaft clamped causing it to stretch and neck down to the required size.
5. Open the tool fully and eject the moulding
6. A post moulding step would be required to cut the end of the needle shaft.

In interesting embodiments, the above processes further comprise mounting a handle to the body portion of the needle.

In still further interesting embodiments, the above processes further comprise anodizing the metal alloy of the hypodermic needle.

### Method of Disposing a Hypodermic Needle

In a further important aspect, the invention relates to a method of disposing a hypodermic needle as described hereinabove, the method comprising the steps of (i) heating at least the tip of said hypodermic needle to a temperature at or above the glass-transition temperature (T_{g}) of said metal alloy, and (ii) deforming said tip of said hypodermic needle so as to blunt said tip.

One of the advantages of the present invention is that hypodermic needles can be disposed in a safe manner. Hence, the tip of the hypodermic needle can be heated by means of readily available heating apparatuses, whereby the tip becomes deformable. Examples of sources for heating the tip to the required temperature, i.e. to a temperature at or above the glass-transition temperature (T_{g}) of said metal alloy, are conventional lighters (e.g. butane lighters), ethanol flames, heating plates for laboratory use, oil baths, etc. In one example, the tip of the hypodermic needle is heated using a common lighter. The needle may then be deformed (moulded) by pushing on to a heat resistant material, e.g. stone or a metal surface so as to blunt the originally sharp edge of the tip.

In particularly relevant embodiments of the above, the hypodermic needle is contaminated with blood, a bodily fluid or a pharmaceutically active ingredient. In such instances, it is of particularly relevance to render the tip of the hypodermic needle blunt whereby perforation of skin can be avoided.

In view of the above, the invention also provides a method of using a hypodermic needle as described hereinabove, the method comprising the step of (i) retracting the hypodermic needle from a mammalian body (e.g. a human body), (ii) heating at least the tip of said hypodermic needle to a temperature at or above the glass-transition temperature (T_{g}) of said metal alloy, and (iii) deforming said tip of said hypodermic needle so as to blunt said tip.

The specifications with respect to the hypodermic needle and the amorphous metal alloy (bulk amorphous alloy) are preferably as described hereinabove.

### EXAMPLES

The following prophetic examples will further illustrate the invention.

### Example 1

A feedstock of Zr_{52.5}Al₁₀Ti₅Cu_{17.9}Ni_{14.6} is heated to the glass transition temperature (410° C.) in an extruder. The feedstock is then extruded into a tube with a diameter of 0.3 mm and an inner bore channel hole of 0.2 mm. The tube is cut into needle size 20 mm. The needle tip is made by grinding the piercing end of the pipe at 3 angles. Finally, the needle tube is attached to a hub.

### Example 2

A feedstock of Zr_{52.5}Al₁₀Ti₅Cu_{17.9}Ni_{14.6} is heated to the glass transition temperature (410° C.) in an injection moulding machine. The alloy is then injected into the mould and using a continuous wire strand as the needle bore channel hole, the needle is formed. The mould is opened and the wire is cut, thus retrieving the needle. The needle tip is made by grinding the piercing end of the pipe at 3 angles. The mould is made so that the hub is an integral part of the needle and thus incorporated into the mould design.

### Example 3

A feedstock of Cu_{47.5}Zr_{47.5}Al₅ is heated to the glass transition temperature (425° C.) in an extruder. The feedstock is then extruded into a tube with a diameter of 0.3 mm and an inner bore channel hole of 0.2 mm. The tube is cut into needle size 20 mm. The needle tip is made by grinding the piercing end of the pipe at 3 angles. Finally, the needle tube is attached to a hub.

### Example 4

A feedstock of Cu_{47.5}Zr_{47.5}Al₅ is heated to the glass transition temperature (425° C.) in an injection moulding machine. The alloy is then injected into the mould and using a continuous wire strand as the needle bore channel hole, the needle is formed. The mould is opened and the wire is cut, thus retrieving the needle. The needle tip is made by grinding the piercing end of the pipe at 3 angles. The mould is made so that the hub is an integral part of the needle and thus incorporated into the mould design.

### Example 5

A feedstock of Pt_{57.5}Cu_{14.7}Ni_{5.3}P_{22.5} is heated to the glass transition temperature (235° C.) In an extruder. The feedstock is then extruded into a tube with a diameter of 0.3 mm and an inner bore channel hole of 0.2 mm. The tube is cut into needle size 20 mm. The needle tip is made by grinding the piercing end of the pipe at 3 angles. Finally, the needle tube is attached to a hub.

### Example 6

A feedstock of Pt_{57.5}Cu_{14.7}Ni_{5.3}P_{22.5} is heated to the glass transition temperature (235° C.) in an injection moulding machine. The alloy is then injected into the mould and using a continuous wire strand as the needle bore channel hole, the needle is formed. The mould is opened and the wire is cut, thus retrieving the needle. The needle tip is made by grinding the piercing end of the pipe at 3 angles. The mould is made so that the hub is an integral part of the needle and thus incorporated into the mould design.

### Example 7

A feedstock of Pd₃₅Cu₃₀Ni₁₀Fe₅P₂₀ is heated to the glass transition temperature (298° C.) In an extruder. The feedstock is then extruded into a tube with a diameter of 0.3 mm and an inner bore channel hole of 0.2 mm. The tube is cut into needle size 20 mm. The needle tip is made by grinding the piercing end of the pipe at 3 angles. Finally, the needle tube is attached to a hub.

### Example 8

A feedstock of Pd₃₅Cu₃₀Ni₁₀Fe₅P₂₀ is heated to the glass transition temperature (298° C.) in an Injection moulding machine. The alloy is then injected into the mould and using a continuous wire strand as the needle bore channel hole, the needle is formed. The mould is opened and the wire is cut, thus retrieving the needle. The needle tip is made by grinding the piercing end of the pipe at 3 angles. The mould is made so that the hub is an integral part of the needle and thus incorporated into the mould design.

### Example 9

A feedstock of Ti_{41.5}Zr_{2.5}Hf₅Cu_{42.5}Ni_{7.5}Si is heated to the glass transition temperature (407° C.) in an extruder. The feedstock is then extruded into a tube with a diameter of 0.3 mm and an inner bore channel hole of 0.2 mm. The tube is cut into needle size 20 mm. The needle tip is made by grinding the piercing end of the pipe at 3 angles. Finally, the needle tube is attached to a hub.

### Example 10

A feedstock of Ti_{41.5}Zr_{2.5}Hf₅Cu_{42.5}Ni_{7.5}Si₁ is heated to the glass transition temperature (407° C.) in an injection moulding machine. The alloy is then injected into the mould and using a continuous wire strand as the needle bore channel hole, the needle is formed. The mould is opened and the wire is cut, thus retrieving the needle. The needle tip is made by grinding the piercing end of the pipe at 3 angles. The mould is made so that the hub is an integral part of the needle and thus incorporated into the mould design.

1. A hypodermic needle composed of a metal alloy, wherein the metal alloy is in a predominantly amorphous form, said amorphous form of said metal alloy having a glass transition temperature (T_{g}) in the range of 50-650° C.

2. The hypodermic needle according to claim 1, wherein the amorphous form of the metal alloy constitutes more than 75% by volume of said metal alloy.

3. The hypodermic needle according to claim 1, wherein the temperature interval, ΔT, between the crystallisation temperature (Tₓ) and the glass transition temperature (T_{g}) of the metal alloy is at least 5 K.

4. The hypodermic needle according to claim 1, wherein the metal alloy is selected from the group consisting of:
a. Copper containing alloys,
b. Platinum containing alloys,
c. Palladium containing alloys,
d. Zirconium containing alloys, and
e. Titanium containing alloys.

5. The hypodermic needle according to claim 4, wherein the metal alloy is selected from the group consisting of:
a. copper containing alloys of the approximate formula CuₓZr_{y}Al_{z}YₚTi_{q}, wherein x=40-70, y=25-55, z=5-10, p=0-5, q=0-5, and the sum x+y+z+p+q is 100, all in atomic percentages;
b. platinum containing alloys of the approximate formula PtₓCu_{y}Ni_{z}Pₚ, wherein x=55-60, y=13-17, z=3-7, p=20-25 and the sum x+y+z+p is 100, all in atomic percentages;
c. palladium containing alloys of the approximate formula PdₓCu_{y}Ni_{z}FeₚP_{q}, wherein x=32-38, y=27-33, z=8-12, p=3-7, q=18-22 and the sum x+y+z+p+q is 100, all in atomic percentages;
d. zirconium containing alloys of the approximate formula ZrₓAl_{y}Ti_{z}CuₚNi_{q}, wherein x=49-56,y=8-12, z=3-7, p=16-20, q=13-17 and the sum x+y+z+p+q is 100, all in atomic percentages; and
e. titanium containing alloys of the approximate formula Ti_{41.5}Zr_{2.5}Hf₅Cu_{42.5}Ni_{7.5}Si₁, all in atomic percentages.

6. The hypodermic needle according to claim 5, wherein the metal alloy is selected from the group consisting of:
a. copper containing alloys of the approximate formula Cu_{47.5}Zr_{47.5}Al₅, all in atomic percentages,
b. platinum containing alloys of the approximate formula Pt_{57.5}Cu_{14.7}Ni_{5.3}P_{22.5}, all in atomic percentages,
c. palladium containing alloys of the approximate formula Pd₃₅Cu₃₀Ni₁₀Fe₅P₂₀, all in atomic percentages,
d. zirconium containing alloys of the approximate formula Zr_{52.5}Al₁₀Ti₅Cu_{17.9}Ni_{14.6}, all in atomic percentages, and
e. titanium containing alloys of the approximate formula Ti_{41.5}Zr_{2.5}Hf₅Cu_{42.5}Ni_{7.5}Si₁, all in atomic percentages.

7. A method of manufacturing a hypodermic needle, the method comprising the steps of
a. providing a feedstock of a molten liquid metal alloy,
b. casting the feedstock into the desired shape in form of a needle while cooling the metal alloys so as to bring said metal alloy into a predominantly amorphous form, said amorphous form of said metal alloy having a glass transition temperature (T_{g}) in the range of 50-650° C., and
c. optionally processing the needle to form a preliminary edge.

8. A method of manufacturing a hypodermic needle, the method comprising the steps of
a. providing a feedstock of a solid piece of a metal alloy, said metal alloy being in a predominantly amorphous form, said amorphous form of said metal alloy having a glass transition temperature (T_{g}) in the range of 50-650° C.,
b. heating the feedstock to or above the glass transition temperature (T_{g}), but below the crystallisation temperature (Tₓ), of said metal alloy,
c. moulding the alloy into the desired shape in form of a needle, and
d. optionally processing the needle to form a preliminary edge.

9. A method according to claim 8, the moulding being conducted by means of an injection moulding machine.

10. A method of disposing a hypodermic needle as defined in claim 1, the method comprising the steps of (i) heating at least the tip of said hypodermic needle to a temperature at or above the glass-transition temperature (T_{g}) of said metal alloy, and (ii) deforming said tip of said hypodermic needle so as to blunt said tip.
